(19) United States
(12) Patent Application Publication
(10) Pub. No.: US 2007/0214015 A1
(43) Pub. Date: Sep. 13, 2007

(54) Healthcare management

(76) Inventor: James G. Christian, Pueblo, CO (US)

Correspondence Address: HANES & SCHUTZ, LLC, 102 SOUTH TEJON ST., SUITE 800, COLORADO SPRINGS, CO, 80903, US

(21) Appl. No.: 11/368,084

(22) Filed: Mar. 6, 2006

(51) Int. Cl. G06F 19/00; G09B 23/28; A61B 5/00

(52) U.S. Cl. 705/3; 600/300; 434/262

(57) ABSTRACT

A computer assessment is performed of ways for managing an individual's health. A patient report is generated, from the computer assessment, for the individual's use. The patient report provides advice tailored to help the individual manage the individual's health. A healthcare provider report is generated, from the computer assessment, for use by the individual's healthcare provider. The healthcare provider report summarizes findings of the assessment and provides counseling guidelines, for the individual's healthcare provider, tailored to the individual and the assessment.

The U.S. Government has a paid-up license in this invention and the right in limited circumstances to require the patent owner to license others on reasonable terms as provided for by the terms of Small Business Innovation and Research grant numbers 1R43DK066711-01A1, 5R44DK060272-3, 2R44DK060272-02, 1R43HD047031-01A1, 2R44DK066711-02, 1R43DK060272-0181 awarded by the U.S. National Institute of Health.

### BACKGROUND OF THE INVENTION

The healthcare industry is continually searching for ways to improve the management of healthcare in individuals. Motivating patients to adopt a healthful treatment plan during brief office visits is a major challenge facing healthcare providers. Often, patients with health related issues, such as disease or health risk factors, need assistance managing their health. The need for assistance may take many forms. For example, the patient may need information, motivation, or help to understand factors related to the patient's condition.

### DESCRIPTION OF THE DRAWINGS

FIG. 1 is a block diagram illustrating one embodiment of the present invention healthcare management system.

FIG. 2 is a flow chart illustrating one embodiment of the present invention method for assisting an individual and the individual's healthcare provider manage the individual's health.

### DETAILED DESCRIPTION OF THE INVENTION

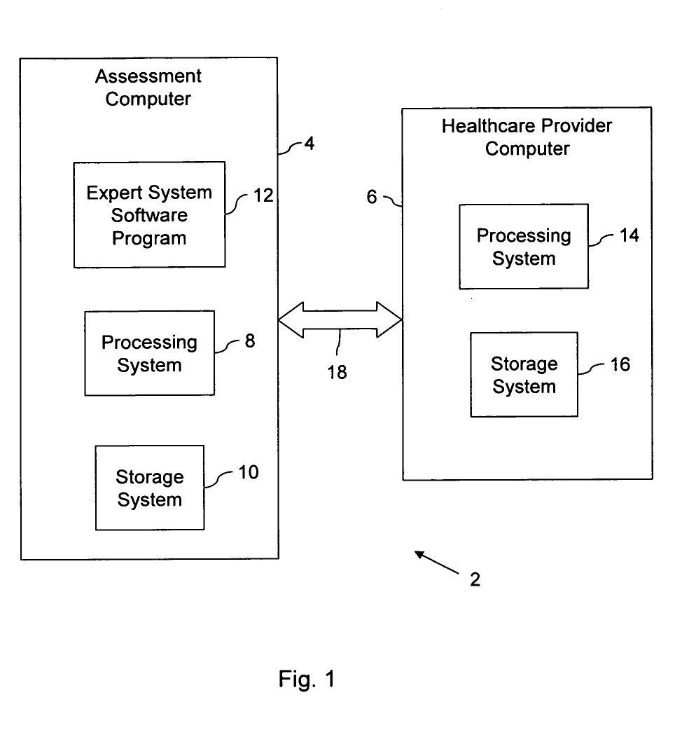

FIG. 1 shows one embodiment of healthcare management system 2 for assisting an individual and the individual's healthcare provider manage the individual's health. In one embodiment, healthcare management system 2 includes assessment computer 4 and healthcare provider computer 6.

Assessment computer 4 is any specific or general purpose computer usable by an individual to perform the steps of the process of the present invention. In one embodiment, assessment computer 4 includes a processing subsystem 8, a storage system 10, and expert system software program 12.

Processing system 8 is any combination of hardware and executable code configured to process software for performing the assessment of the present invention process.

Storage system 10 is any device or system configured to store data or executable code. Storage system 10 may also be a program storage system tangibly embodying a program, applet, or instructions executable by processing system 8 for performing the method steps of the present invention executable by processing system 8. Storage system 10 may be any type of storage media such as magnetic, optical, or electronic storage media.

Storage system 10 is illustrated in FIG. 1 as a single device. Alternatively, storage system 10 may include more than one device. Furthermore, each device of storage system 10 may be embodied in a different media type. For example, one device of storage system 10 may be a magnetic storage media while another device of storage system 10 is an electronic storage media.

Expert system software program 12 is any type of executable code which, when executed, performs the method steps of the present invention process. Expert system software program 12 may be stored in storage system 10 and processed in processing system 8.

Healthcare provider computer 6 is any specific or general purpose computer usable by a healthcare provider to receive a report in electronic, optical, or magnetic form. In one embodiment, healthcare provider computer 6 includes a processing subsystem 14 and a storage system 16.

Processing system 14 is any combination of hardware and executable code configured to process software for performing the assessment of the present invention process.

Storage system 16 is any device or system configured to store data or executable code. Storage system 16 may also be a program storage system tangibly embodying a program, applet, or instructions executable by processing system 14 for performing the method steps of the present invention executable by processing system 14. Storage system 16 may be any type of storage media such as magnetic, optical, or electronic storage media.

Storage system 16 is illustrated in FIG. 1 as a single device. Alternatively, storage system 16 may include more than one device. Furthermore, each device of storage system 16 may be embodied in a different media type. For example, one device of storage system 16 may be a magnetic storage media while another device of storage system 16 is an electronic storage media.

In one embodiment, assessment computer 4 and healthcare provider computer 6 communicate over a network 18. Network 18 may be any type of network such as a local area network, a wide area network, or the Internet. Communication over a network 18 allows assessment computer 4 to be able to send or dispatch a healthcare provider report to healthcare provider computer 6.

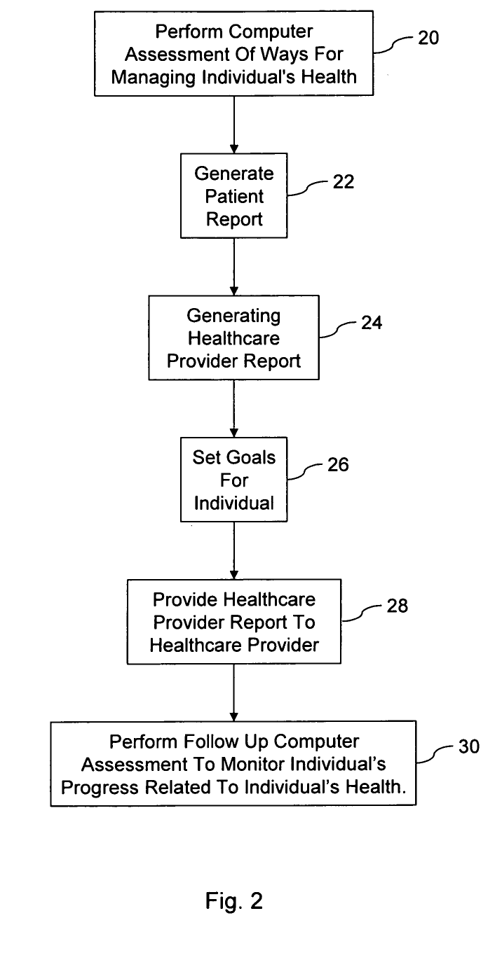

FIG. 2 is a flow chart representing steps of one embodiment of the present invention. Although the steps represented in FIG. 2 are presented in a specific order, the present invention encompasses variations in the order of steps. Furthermore, additional steps may be executed between the steps illustrated in FIG. 2 without departing from the scope of the present invention.

A computer assessment is performed 20 of ways for managing the individual's health. In one embodiment, performing 20 a computer assessment of ways for managing the individual's health includes the individual interacting with an expert system software program to assess ways the individual can better manage the individual's health.

There are several ways that the individual can perform the computer assessment. The expert system software can be accessed in a clinic setting on a stand alone PC or through the clinic intranet. The software can also be accessed through the Internet.

A patient report is generated 22, from the computer assessment, for the individual's use. The patient report provides advice tailored to help the individual manage the individual's health. In one embodiment, the patient report is generated real-time during the computer assessment session and available to the patient via a PDF file or printed out.

A healthcare provider report is generated 24, from the computer assessment, for use by the individual's healthcare provider. The healthcare provider report summarizes findings of the assessment and provides counseling guidelines, for the individual's healthcare provider, tailored to the individual and the assessment. This report provides a brief, bulleted summary of the findings from the individual's assessment (e.g., patient perceptions of risk, priority change areas, perceived barriers to change), and provides the healthcare provider with counseling guidelines specifically for the individual based upon evidence for effective physician motivational counseling. The healthcare provider report can be emailed directly to the healthcare provider or printed by the individual and then directly provided to the healthcare provider by the individual. Alternative, the individual may choose to completely self-manage the disease or health risks and not discuss the self-management plan with a healthcare provider.

The individual reviews the patient report and is encouraged to complete 26 a simple goal setting process which is focused on issues identified in the assessment process. The individual sets 26 goals related to the assessment.

The process is designed to encourage the individual to provide 28 the individual's healthcare provider with the healthcare provider report and discuss the individual's goals for managing a disease(s) and/or reducing risk with the healthcare provider. In one embodiment, the healthcare provider report is provided 28 to the individual's healthcare provider.

A follow up computer assessment is performed 30 to monitor the individual's progress related to the individual's health. The computer assessment can be performed 30 again at later times and has the capability to measure movement (i.e. an individuals' progress) toward meeting the individual's goals and assessing new subsequent goals.

The system and method of the present invention can be used to manage existing diseases, injuries, or conditions, to prevent a disease, or to reduce risks of health-related complications (e.g. risks to mother and baby resulting from premature delivery of a baby. Content of the expert system software can address diseases including diabetes, heart diseases (including primary and secondary cardiovascular disease prevention, congestive heart failure), chronic obstructive pulmonary disease, osteoporosis, depression as well as preventing a preterm birth or low birth weight baby and management of blood pressure, management of blood lipids, weight management, smoking cessation, adherence to medications, and management of orthopedic injuries and conditions.

The system and method of the present invention are well suited for more complex situations where combinations of drugs would optimize patient health or multiple risks need to be addressed by the patient and the patient's physician. An example related to cardiovascular diseases and/or diabetes might be when a patient may benefit from taking a statin to lower LDL cholesterol, one or more antihypertensive agents, aspirin, and possibly folic acid. In these types of situations more detailed risk assessment, assessment of side effects and drug tolerance issues, and patient adherence concerns need to be addressed. The system and method also support cases where a single polypill (which contains a statin, antihypertensive agent(s), aspirin and possibly folic acid in some potential formulations) is used—for even though the patient can get multiple pharmacotherapies from a single pill, the assessment and drug tolerance issues are still complex with a polypill. The system and method of the present invention support effective and efficient patient self management of compliance and physician communications with patient.

Similarly, pregnant women who have a high risk for preterm birth can reduce these risks by stopping illicit drug, tobacco and alcohol use (if present), getting treatment for depression if needed, improving nutrition, gaining and adequate amount of weight during the pregnancy and living in an environment where risks of physical harm are reduced. Physicians and midwives can reduce risks of preterm birth in the patient by screening for and treating several infections (if present in the patient). The system allows patients and clinicians to quickly and easily assess these risks and provide tailored advice to the patient and counseling tips to the physician and/or midwife, support goal setting and patient self management.

In addition, people who have an orthopedic injury or condition can better understand how to care for their injury or condition.

Variations of this computer-assisted system can be used by individuals who range in age from younger children (approximately 8 years old and up), adolescents, young adults, middle aged adults and adults over 75 years in age.

There are many advantages to the present invention system and method. The system and method provide a platform that, in some applications, can be used as a base to increase the consistency with which healthcare providers discuss disease management plans with individuals who use this system.

The combination of assessment, tailored expert system feedback (based on several behavioral models that are well established and in wide use in the healthcare industry), incorporation of a clear goal setting process, and the potential to utilize healthcare providers to counsel individuals using the system, to reinforce and support the individuals' self-management goals and initiatives is more effective than if these actions are done separately.

The healthcare provider report provides professionally written counseling guidelines to potentially enhance the quality of counseling provided by healthcare providers. The system and method provide a focused format so that all elements can be completed in a brief time period, thus enabling healthcare providers to actually use this system in a real-world clinical setting.

The system and method are capable of assessing more complex patient risk profiles where more detailed assessment of multiple physiologic profiles is required (i.e. when a physician is assessing appropriateness of prescribing a polypill). In these situations physicians must also assess side effect history for one or more drugs before prescribing a polypill. Closer monitoring of the effects of the polypill on blood pressure and cholesterol levels as well as tracking drug tolerance is required. The computer-based system can address these complex issues and support both patient and physician over the course of multiple clinic visits and ongoing treatment.

The foregoing description is only illustrative of the invention. Various alternatives and modifications can be devised by those skilled in the art without departing from the invention. Accordingly, the present invention embraces all such alternatives, modifications, and variances that fall within the scope of the appended claims.

What is claimed is:

1. A method for assisting an individual and the individual's healthcare provider manage the individual's health, the method comprising:
performing a computer assessment of ways for managing the individual's health;
generating a patient report, from the computer assessment, for the individual's use, the patient report providing advice tailored to help the individual manage the individual's health; and
generating a healthcare provider report, from the computer assessment, for use by the individual's healthcare provider, the healthcare provider report summarizing findings of the assessment and providing counseling guidelines, for the individual's healthcare provider, tailored to the individual and the assessment.

2. The method of claim 1 wherein performing a computer assessment of ways for managing the individual's health includes the individual interacting with an expert system software program to assess ways the individual can better manage the individual's health.

3. The method of claim 1 further including the individual setting goals related to the assessment.

4. The method of claim 1 further including providing the healthcare provider report to the healthcare provider.

5. The method of claim 1 further including performing a follow up computer assessment to monitor the individual's progress related to the individual's health.

6. A computer process for assisting an individual and the individual's healthcare provider manage the individual's health, the computer process comprising:
performing an assessment of ways for managing the individual's health;
generating a patient report, from the computer assessment, for the individual's use, the patient report providing advice tailored to help the individual manage the individual's health; and
generating a healthcare provider report, from the computer assessment, for use by the individual's healthcare provider, the healthcare provider report summarizing findings of the assessment and providing counseling guidelines, for the individual's healthcare provider, tailored to the individual and the assessment.

7. The computer process of claim 6 wherein performing a computer assessment of ways for managing the individual's health includes executing an expert system software program to assess ways the individual can better manage the individual's health.

8. The computer process of claim 6 further including the dispatching the healthcare provider report to the individual's healthcare provider.

9. The computer process of claim 6 further including performing a follow up assessment to monitor the individual's progress related to the individual's health.

10. A program storage system readable by a computer, tangibly embodying a program, applet, or instructions executable by the computer to perform method steps for assisting an individual and the individual's healthcare provider manage the individual's health, the method steps comprising:
performing an assessment of ways for managing the individual's health;
generating a patient report, from the computer assessment, for the individual's use, the patient report providing advice tailored to help the individual manage the individual's health; and
generating a healthcare provider report, from the computer assessment, for use by the individual's healthcare provider, the healthcare provider report summarizing findings of the assessment and providing counseling guidelines, for the individual's healthcare provider, tailored to the individual and the assessment.

11. The program storage system of claim 10 wherein performing a computer assessment of ways for managing the individual's health includes executing an expert system software program to assess ways the individual can better manage the individual's health.

12. The program storage system of claim 10 further including the dispatching the healthcare provider report to the individual's healthcare provider.

13. The program storage system of claim 10 further including performing a follow up assessment to monitor the individual's progress related to the individual's health.
